(19) United States
(12) Patent Application Publication
(10) Pub. No.: US 2021/0366607 A1
(43) Pub. Date: Nov. 25, 2021

(54) HEALTH SCREENING KIOSK

(71) Applicant: Bipin KOLLURI, Fairview, TX (US)

(72) Inventor: Bipin KOLLURI, Fairview, TX (US)

(21) Appl. No.: 17/325,035

(22) Filed: May 19, 2021

Related U.S. Application Data
(60) Provisional application No. 63/026,784, filed on May 19, 2020.

(51) Int. Cl. G16H 40/67; G16H 50/30; A61B 5/15; G06Q 20/18; G06Q 20/20

(52) U.S. Cl. CPC G16H 40/67; G16H 50/30; G06Q 20/209; G06Q 20/18; A61B 5/150854

(57) ABSTRACT

This invention relates to systems and methods for identifying subjects infected by viruses. Present invention is particularly directed to providing systems and methods that allow secure distribution of self-expiring health badges through a kiosk to the subjects free of Severe acute respiratory syndrome coronavirus 2 (SARS-CoV-2) infection. The kiosk includes a customer interface which comprises (a) a computer system; (b) a finger slot for collecting subject's blood sample and rapid testing, and (c) a badge/card printer and an output slot. The kiosk is located at a plurality sites and includes a camera to capture an image of the subject and attaches the image to self-expiring badge after the subjects are tested negative for SARS-CoV-2 infections. The self-expiring badge uses a time indicator and provides a clear indication of expiration of the badge after a predetermined period interval. Furthermore, the badge acts to prevent unauthorized usage and are re-usable.

### CROSS-REFERENCE TO RELATED APPLICATIONS

This non-provisional application claims benefit of provisional application U.S. Ser. No. 63/026,784 filed May 19, 2020, the entirety of which is hereby incorporated by reference.

### BACKGROUND OF THE INVENTION

### Field of the Invention

The present invention generally relates to the fields of medicine and molecular biology and particularly related to screening methods and systems for microorganism infections. Present invention is more particularly related to systems, kits, devices, and methods for assessing the health status of a person and secure distribution of health badges to the infection-free subjects.

### BRIEF SUMMARY OF THE INVENTION

The present invention is directed to systems and methods for identifying subjects infected by viruses. Present invention is particularly directed to providing systems and methods that allow secure distribution of self-expiring health badges after the subjects are tested negative for Severe acute respiratory syndrome coronavirus 2 (SARS-CoV-2) infection.

Thus, in one embodiment of the present invention, there is provided a self-service kiosk for producing self-expiring badge comprising: a customer interface with a computer system; a collector for collecting a biological sample from a subject; an analyzer for analyzing the biological sample; and a self-expiring badge printer and output; wherein the analyzer analyzes the biological sample for microorganism infections and wherein said self-expiring badge is issued to said subject upon said subject tests negative for microorganism infections.

In another embodiment of the present invention, there is provided a non-transitory computer-readable medium storing program code that causes a processor to execute operations, at a self-service kiosk, comprising: displaying tests available to be performed at the kiosk; obtaining from a selection of a test from a subject; obtaining payment from the subject and performing financial transactions; performing the selected test; providing results of the test to the subject; and printing a self-expiring badge, wherein the self-expiring badge is printed upon the subject tests negative for microorganism infections.

In yet another embodiment of the present invention, there is provided a method for identifying subjects free of microorganism infections using a kiosk comprising the steps of: displaying instructions for using the kiosk; collecting a blood sample from the subject; testing the blood sample for the microorganism infections and producing a result; storing the result for subsequent retrieval and transmission; and distributing a self-expiring identification badge upon said subject tests negative for microorganism infections.

While various aspects of the present invention have been particularly shown and described with reference to the exemplary, non-limiting, embodiments above, it will be understood by those skilled in the art that various additional aspects and embodiments may be contemplated without departing from the sprit and scope of the present invention.

### BRIEF DESCRIPTION OF THE DRAWINGS

FIG. 1 is a block diagram of the system architecture of the platform of the present invention.

FIG. 2 is the block diagram of a kiosk of the present invention.

FIG. 3 is a flow chart illustrating the algorithm implemented by the kiosk software.

FIG. 4 is a rapid-test cassette used in the present invention.

FIG. 5 is a front view of a self-expiring badge/card of the present invention.

FIG. 6 is an exemplary kiosk of the present invention.

FIG. 7 is an alternative kiosk of the present invention.

### DETAILED DESCRIPTION OF THE INVENTION

As used herein the specification, “a” or “an” may mean one or more. As used herein, the terms “subject”, “customer” and “user” may refer to a mammalian species, frequently refer to a human subject. Throughout the invention, the terms subject, customer and user are used interchangeably. As used herein the term “sample” refers to anything which may contain an analyte for which an analyte assay is desired. The sample may be a biological sample, such as a biological fluid. Examples of biological fluids include blood, plasma, serum, saliva, mucus, tears, and semen. Other biological samples (e.g., urine, stool) can also be contemplated. As used herein, the term “antigen” refers to a target molecule that is specifically bound by an antibody through its antigen recognition site. The antigen may be monovalent or polyvalent, i.e., it may have one or more epitopes recognized by one or more antibodies. Examples of kinds of antigens that can be recognized by antibodies include polypeptides, oligosaccharides, glycoproteins, polynucleotides, lipids, etc.

As used herein, microorganisms can include any of the viruses, bacteria, yeast, or parasites.

The viruses can include but not limited to Adenovirus, Adenovirus F40/41, Astrovirus, Coronavirus, Coronavirus 229E, Coronavirus HKU1, Coronavirus NL63, Coronavirus OC43, Cytomegalovirus (CMV), Enterovirus, Herpes simplex virus 1 (HSV-1), Herpes simplex virus 2 (HSV-2), Human herpesvirus 6 (HHV-6), Human Metapneumonvirus, Human Metapneumovirus, Human parechovirus, Human Rhinovirus/Enterovirus, Influenza A, Influenza A/H1, Influenza A/H1-2009, Influenza A/H3, Influenza B, Middle East Respiratory Syndrome Coronavirus, Norovirus GI/GII, Parainfluenza virus, Parainfluenza Virus 1, Parainfluenza Virus 2, Parainfluenza Virus 3, Parainfluenza Virus 4, Respiratory Syncptial Virus, Respiratory Syncytial Virus, Rotavirus A, Sapovirus (I, II, IV and V), Severe acute respiratory syndrome coronavirus 2 (SARS-CoV-2), and Varicella zoster virus (VZU).

The bacteria can include but not limited to E. coli 0157, Chlamydia pneumoniae, E. coli (STEC) stx1/stx2, Enteroaggregative E. coli (EAEC), Enterobacter cloacae complex, Enteropathogenic E. coli (EPEC), Enterotoxigenic E. coli (ETEC) lt/st, Escherichia coli, Klebsiella oxytoca, Klebsiella pneumoniae, Legionella pneumophila, Mycoplasma pneumoniae, Proteus, Shiga-like toxin-producing, Shigella/Enteroinvasive E. coli (EIEC), Streptococcus pneumoniae, Streptococcus pyogenes, Acinetobacter baumannii, Acinetobacter calcoaceticus-baumannii complex, Antimicrobial Resistance Genes CTX-M, Antimicrobial Resistance Genes IMP, Antimicrobial Resistance Genes KPC, Antimicrobial Resistance Genes mecA/C and MREJ, Antimicrobial Resistance Genes NDM, Antimicrobial Resistance Genes Oxa48-like, Antimicrobial Resistance Genes VIM, Atypical Bateria, Bordetella parapertussis, Bordetella pertussis, Campylobacter (jejuni, coli and upsaliensis), Chlamydia (Chlamydophila) pneumoniae, Clostridium (toxin A/B), Diarrheagenic E. coli/Shigella, Enerobacteriaceae, Enterobacter aerogenes, Enterococcus, Entrogacter cloacae, Escherichia coli, Escherichia coli K1, Haemophilus influenza, Haemophilus influenzae, Heamophilus influenza, Klebsiella oxytoca, Klebsiella pneumoniae group, KPC-carbapenem resistance, Listeria monocytogenes, mecA-methicillin resistance, Morazella catarrhalis, Mycoplasma pneumonia, Neisseria meningitidis, Neisseria meningtidis, Plesiomonas shigellodies, Proteus spp., Pseudomonas aeruginosa, Quanlitative Bacteria, Salmonella, Semi-Quantitative Bacteria, Serratia marcescens, Staphylococcus aureus, Steptococcus agalactiae, Stetococcus pneumoniae, Streptococccus phenumoniae, Streptococcus agalactiae, Streptococcus pyogenes, vanA/B-vancomycin resistance, Vibrio (parahaemolyticus, vulnificus and cholerae), Vibrio cholerae, Yersinia enterocolitica, Enterococcus-Listeria monocytogenes, Staphylococcus-Staphylococcus aureus, Streptococcus-Streptococcus agalactiae, Streptococcus pneumoniae, Streptococcus pyogenes, Campylobacter (jejuni, coli and upsaliensis), Clostridium (toxin A/B), Plesiomonas shigellodies, Salmonella, Yersinia enterocolitica, Vibrio (parahaemolyticus, vulnificus and cholerae). Vibrio cholerae, Diarrheagenic E. coli/Shigella-Enteroaggregative E. coli (EAEC), Enteropathogenic E. coli (EPEC), Enterotoxigenic E. coli (ETEC) lt/st, Shiga-like toxin-producing, E. coli (STEC) stx1/stx2, E. coli 0157, and Shigella/Enteroinvasive E. coli (EIEC).

The yeast can include but not limited to Candida albicans, Candida glabrata, Candida krusei, Candida parapsilosis, Candida tropicalis, and Cryptococcus neoformans/gatti.

The parasites can include but not limited to Cryptosporidium, Cyclospora cayetanensis, Entamoeba histolytica, and Giardia lamblia.

Exemplary, non-limiting embodiments of the present invention are discussed in detail below. While specific configurations, methods and drawings are discussed to provide clear understanding, it should be understood that the specific embodiments and their respective configurations are provided for illustration purposes only. A person skilled in the relevant art will recognize that other configurations may be used without departing from the spirit and scope of the invention.

### General Embodiments of the Invention

Present invention is related to systems, kits, devices, and methods for assessing the health status of a person and providing self-expiring health badges to the infection-free subjects. The devices, kits, and systems for health screening and secure distribution of self-expiring health badges can be used at any suitable location. In preferred embodiments, the devices, kits, and systems for assessing subjects' exposure to SARS-CoV-2 and providing self-expiring health badges to the infection-free subjects can be used at a point of presence. Exemplary point of presence can be a retail location or a store, e.g., a pharmacy store or a supermarket, malls, schools, airports, public places (e.g. parks), a medical office, a clinical laboratory, a health station, a gym, or any heavy traffic moving locations that are convenient and easily accessible.

The devices, kits, and systems can conduct a SARS-CoV-2 infection test and issue a self-expiring health badges to infection free subjects within any suitable time period, e.g. in about 1 hour, 50 minutes, 40 minutes, 30 minutes, 20 minutes, 10 minutes, or 5 minutes, from the time a sample is obtained from the subject.

The present devices, kits, and systems for health screening can further comprise a means for assessing an additional health indicator of the subjects. Any suitable means can be used. In some embodiments the means is used to assess at least one of weight (body mass index), blood pressure, temperature, heart rate and oxygen saturation.

Further, according to the present embodiment, the system comprises a display device where the display device is a touch screen display such that customers can easily touch the appropriate portion of the screen in response to various instructions and/or queries.

Exemplary advantages of a system in accordance with the present system include allowing subjects to avoid frequent visits to doctor's office for rapid testing which could be costly and inconvenient.

### User Registration

When subject enters a facility that is equipped with a system according to the present invention, the subject is greeted by a number of individual self-service kiosks, each spaced apart from one another in order to afford the subjects a certain level of privacy as he or she may answer personal questions displayed on the screen.

According to the present embodiment, the subject is instructed, via the display device to enter the information to complete the registration process. Any suitable means for proper identification and registration can be used. For example, typical registration process comprises verification of the subject's demographical information, e.g. name, address, phone number, employer information, valid photo identification card and sign the forms. Other methods such as several questionnaires can also be used.

In a most preferred process, the subjects can complete the registration and validation prior to arriving at the kiosk. The subjects can download the fast-trac app on smart device (e.g., iOS, android, windows) or go to the online website and complete the registration process prior to arriving at the kiosk. The subject may be prompted to upload a valid identification card and a photo (selfie) for validation. After successful pre-registration and validation, subjects can determine the nearest kiosk location for subsequent health screening and receiving the self-expiring health badges. For the subjects who completes the pre-registration and validation, the user interface at the kiosk provides a means to populate the pre-registered information and directs the subjects with the next steps. The subjects can set up a kiosk appointment before arriving at the kiosk facility. Additionally, if the subjects are paying for the tests, the payments and financial transactions can be processed through the fast-trac app. The users can pay with Fiat currency or crypto currency.

The present invention contemplates using a stand-alone self-service (self-help) registration system for completing the registration process. A self-service registration machine includes a computer system that includes a touch screen for receiving input information and completing the registration and validation process. The present system may further comprise a means for assessing an identifier of the subjects. Any suitable means can be used. In some embodiments the means is an iris scan, a fingerprinting device, a means for assessing voice identification, photograph, electronic signature, or photo identification. In all the embodiments and aspects of the present invention, the user data is stored securely on the cloud. The cloud service that provides such a service generally stores customer data remotely from the premises of the customer.

### Rapid Tests

Self-service kiosks of present invention hold the rapid test cassettes and buffer solutions. Thus, present disclosure provides kits for assessing subjects' exposure to SARS-CoV-2. The kit comprises rapid test devices, said rapid test devices are configured to assess analytes in a sample derived from the subjects. The analytes are selected from the group consisting of SARS-CoV-2 antigen, a SARS-CoV-2 polynucleotide, and an anti-SARS-CoV-2 antibody.

In one aspect, the present disclosure provides a test device for assessing subjects' exposure to SARS-CoV-2. The devices are configured to assess analytes in a sample derived from the subjects. The analytes are selected from the group consisting of SARS-CoV-2 antigen, a SARS-CoV-2 polynucleotide, an anti-SARS-CoV-2 antibody.

In another aspect, the present disclosure provides a system for assessing subjects exposure to SARS-CoV-2, which system comprises any of the above kits and devices.

In yet another aspect, the present disclosure provides a method for assessing subjects' exposure to SARS-CoV-2. The devices are configured to assess analytes in a sample derived from the subjects. The analytes are selected from the group consisting of SARS-CoV-2 antigen, a SARS-CoV-2 polynucleotide, an anti-SARS-CoV-2 antibody.

In some embodiments, the present system can further comprise a means for obtaining at least two different types of samples from the subjects. In other embodiments, the present system can further comprise a means for collecting multiple volumes of blood samples to run multiple assays. The device can make at least two punctures at once to increase the amount of blood sample volume needed to perform multiple assays on the blood sample.

In an embodiment, if the customer is tested positive for SARS-CoV-2 infection, the system provides instructions for the next course of action. For example, the customer can schedule an appointment with the physician for further examination and diagnosis (e.g. PCR testing). The positive results data may be shared with customer's physician or any assigned physician. All the customer data (excluding the private data) can be stored into Centers for Disease Control and Prevention (CDC) or National Contact Tracing Database.

### Respiratory Monitor

Self-service kiosk of present invention includes a respiratory monitor capable of pulmonary function testing. In one embodiment, the respiratory monitor incorporates nitric oxide detectors where exhaled breadth from a customer enters the detection device and a NOₓ sensor measures the NOₓ concentration in the sample. Importantly, the sensors provide an accurate concentration of nitric oxide in the presence of other possibly interfering gas components (e.g. water, carbon dioxide). In one aspect, the respiratory monitor measures the concentration of fractionally exhaled nitric oxide (FeNO). Importantly, the sensors provide an accurate concentration of nitric oxide in the presence of other possibly interfering gas components (e.g. water, carbon dioxide). In one aspect, the customer exhales into the device through a mouthpiece wherein the mouthpiece allows the customer to inhale outside air through the mouthpiece, and exhale through the mouthpiece and into the device through a path separate from the path of inhaled air. In a related aspect, the mouthpiece can be a single use, disposable component that can be sanitized prior to use or easily sanitizable and reusable. In all the aspects of the embodiment, the respiratory monitor may comprise one or more humidity equilibrators to equilibrate the humidity of the breath sample. In one aspect, additional filters can be incorporated into the device for filtering out microorganisms (e.g. bacteria, fungi, virus).

### Self-Expiring Badge

After tested negative for SARS-CoV-2 infections, the subjects are issued self-expiring identification badges. Thus, self-service kiosks of present invention are equipped with self-expiring badge printers. In a most preferred object of this invention is to provide a self-expiring photographic badge that expires after a predetermined time interval. Another object of this invention is to provide a Badge, the expiration of which may be visually observed. Another object of this invention is to provide a badge which is simple to use. Another object of this invention is to provide a health badge which acts to prevent unauthorized use of the badge. Another object of this invention is to distribute the badge that is fail-safe. Yet another object of this invention is to provide a Badge which may be reused. Still another object of this invention is to provide the badge that is tamper resistant. Present disclosure is directed to make all the data secure by exploring various secure mechanisms. In a non-limiting example, it is contemplated to use blockchain technology.

In a preferred embodiment, the present invention provides system and methods for continuously producing a plurality of self-expiring photographic badges. The system and methods enable the subjects to create the self-expiring photographic badge for themselves after testing negative for SARS-CoV-2 infections. In one aspect, the self-expiring identification badge expires after a predetermined time interval. In another aspect, the expiration of the badge may be visually observed. In another aspect, the health badge acts to prevent unauthorized use of the badge. In yet another aspect, the badge is re-usable.

In accordance with a further aspect of the invention, the system permits subjects patients retrieve information regarding future health screening and badge expiration dates. If the badge has been expired, the customer can complete the health screening and re-validate the same badge. Thus, the kiosk includes a card reader or scanner, and the customer is instructed, via the display device, to slide, scan or insert the badge in a slot. The data read from the card uniquely identifies the customer by using a unique alpha-numeric combination or some other type that corresponds only to the one individual customer. The data can be stored on the card in a magnetic strip, barcode, or any other suitable format for storing data. Only after the identification of the person corresponding to the data on the card has been established can any personal information be accessed. Accordingly, each customer's privacy, personal health screening data, or otherwise sensitive data, is not accessible or displayed at the kiosk display device until the patient's identification is made based on data read from the card.

### Exemplary Systems of the Invention

In a preferred embodiment, there is a self-service kiosk for producing self-expiring badge comprising: a customer interface with a computer system; a collector for collecting a biological sample from a subject; an analyzer for analyzing the biological sample; and a self-expiring badge printer and output; where the analyzer analyzes the biological sample for microorganism infections and where the self-expiring badge is issued to the subject upon the subject tests negative for microorganism infections. In one aspect, the customer interface with a computer system provides test results of analyzing the biological sample. In a related aspect, the microorganism is a virus and said self-expiring badge is issued to said subject upon said subject tests negative for viral infections. In a related aspect, the microorganism is a bacterium and said self-expiring badge is issued to said subject upon said subject tests negative for bacterial infections. In a related aspect, the microorganism is a yeast and said self-expiring badge is issued to said subject upon said subject tests negative for yeast infections. In a related aspect, the microorganism is a fungus and said self-expiring badge is issued to said subject upon said subject tests negative for fungal infections. In one aspect, the collector comprises a finger stick device for obtaining the biological sample from the subject. In one aspect, the biological sample is blood. In a preferred aspect, the expiration of the self-expiring badge is visually observed. In a preferred aspect, the self-expiring badge includes an area wherein the expiration date of the badge is written. In one aspect, the self-expiring badge is tampering resistant. In one aspect, the self-expiring badge is re-usable.

In one embodiment, the self-service kiosk of the present invention further comprising a breathalyzer for receiving and testing a breath sample from the subject.

In one embodiment, the self-service kiosk of the present invention further comprising a card reader for obtaining payment information from the subject.

In one embodiment, the self-service kiosk of the present invention further comprising an Internet connection hub for transmission of the test results to a cloud storage or a user device.

In a preferred embodiment, there is a non-transitory computer-readable medium storing program code that causes a processor to execute operations, at a self-service kiosk, comprising: displaying tests available to be performed at the kiosk; obtaining from a selection of a test from a subject; obtaining payment from the subject and performing financial transactions; performing the selected test; providing results of the test to the subject; and printing a self-expiring badge, where the self-expiring badge is printed upon the subject tests negative for microorganism infections. In one aspect of the non-transitory computer-readable medium, where the available tests include a biological sample analysis test that is performed automatically by collecting the biological sample from the subject. In one aspect of the non-transitory computer-readable medium, where the available tests further comprise a non-invasive test for vitals measurements. In one aspect of the non-transitory computer-readable medium, where the non-invasive test measures at least one of weight (body mass index), blood pressure, heart rate and oxygen saturation. In one aspect of the non-transitory computer-readable medium, where the program code causes the processor to execute a further operation comprising transmitting the results of the test to a mobile application on a user device. In an alternative aspect of the non-transitory computer-readable medium, where the program code causes the processor to execute further operations comprising at least one of providing a guidance plan to the subject based on the test results and sharing the test results with subject's physician or any assigned physician.

In a preferred embodiment, the present invention provides a method for identifying subjects free of microorganism infections using a kiosk comprising the steps of: displaying instructions for using the kiosk; collecting a blood sample from the subject; testing the blood sample for the microorganism infections and producing a result; and storing the result for subsequent retrieval and transmission; and issuing a self-expiring identification badge upon said subject tests negative for microorganism infections. In one aspect of the method, the kiosk includes the step of registering and authorizing the subject to access the kiosk. In one aspect of the method, expiration of the self-expiring badge is visually observed. In one aspect of the method, the self-expiring badge includes an area wherein the expiration date of the badge is written. In one aspect of the method, the self-expiring badge is tampering resistant.

In a related embodiment there is a non-transitory computer-readable medium storing program code that causes a processor to execute operations, at a self-service kiosk, comprising: the step of capturing a digital still photograph of the subject interacting with the kiosk.

The present invention is directed to achieve the following goals:
develop a device for easy access,
help expedite the testing process throughout the country,
establish contact tracing, help subjects with last 2 weeks of touch points,
develop an app to help customers with faster processing,
manufacture devices (kiosks) at affordable cost and scale,
distribute the kiosks at various key locations throughout the country,
hake the devices secure, hack proof and tamper proof,
make the data secure, may be on a blockchain technology,
private data storage and shared only with customer's physician or assigned, physician or pharmacy of positive results to schedule an appointment with the doctor for PCR test etc.,
positive results data from lab/pharmacy/doctor to synchronize with the app/device, and
all the data (except private data of customers) to store into Centers for Disease Control and Prevention (CDC) or National Contact Tracing Database.

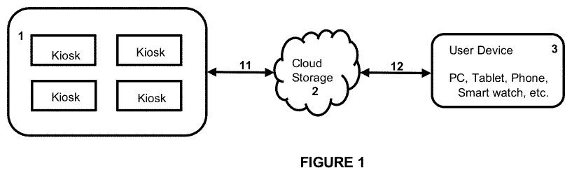

As shown in the FIG. 1, the platform of the present invention includes three sub-systems interconnected through the internet: kiosk(s) 1; cloud storage and infrastructure 2; and user device 3 such as a personal computer (PC), tablet computer, smart phone or smart watch. Kiosk 1 and cloud storage 2 are connected by Internet connection 11 and user device 3 are connected by Internet connection 12.

Kiosk 1 is used by a user to complete or import the registration information and collect biological samples. Kiosk 1 is preferably placed in high traffic venues such as a retail location or a store, e.g., a pharmacy store or a supermarket, malls, schools, airports, public places (e.g. parks), a medical office, a clinical laboratory, or a health station, or any heavy traffic moving locations that are convenient and easily accessible. The user can input the data into kiosk 1 via a keyboard/mouse or touchscreen, or alternatively, may input remotely from user device 3 via internet connection 12 with cloud storage 2. The data obtained by kiosk 1 is provided to cloud storage 2 via Internet connection 11. In addition, an attendant may optionally be present at kiosk 1 in order to assist the users operating kiosk 1, and to maintain and service the kiosk.

Kiosk 1 includes a blood or fluid collector that collects blood/fluid from the kiosk user into a container. An example of such a blood/fluid collector includes, without limitation, a finger stick device that pricks the finger of the kiosk user. Kiosk 1 retrieves the container for analysis and measurement of the collected blood/fluid. The Kiosk 1 may also include a tissue collector (e.g. hair).

Optionally, Kiosk 1 may also be equipped with a set of non-invasive test devices that can be used by the kiosk user to import vitals measurement data. Examples of such devices include, without limitation, weight scales, heart rate monitors/oxymeters and blood pressure devices.

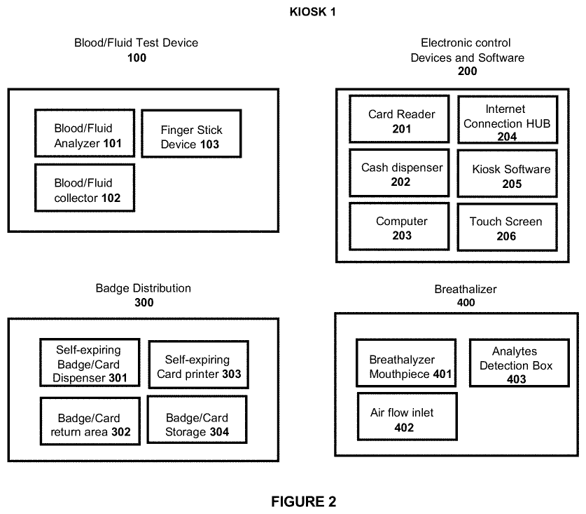

As shown in the FIG. 2, the blood/fluid test devices 100 of kiosk 1 may include, without limitation, a blood/fluid collection device 103 that draws a small amount of blood or fluid from kiosk user when the user puts the finger in the finger slot and presses the button to initiate the process; a blood/fluid collector 102 for collecting the blood/fluid collected from kiosk user; a blood/fluid analyzer 101 into which collector 102 is automatically placed. Kiosk 1 may include one or more of each of collection device 103, collector 102, and analyzer 101. When present, the optional attendant such as a qualified health processional may assist the kiosk user in using collection device 103 and placing the collected blood/fluid into collector 102 and analyzer 101. Additionally, Kiosk 1 includes a safe disposal into which contaminated medical supplies are safely disposed.

The electronic control devices and software 200 of kiosk 1 may include, without limitation, card reader 201; computer 203 including a processor; Internet connection hub 204; touch screen 206; and kiosk software 205. The kiosk user interacts with kiosk 1 via touch screen 206, with or without the help of optional attendant. The kiosk user identifies himself or herself via touch screen 206 and is guided through the process of performing selected tests by touch screen 206 and kiosk software 205. Though kiosk 1 may be used anonymously, some form of contact information such as an email address or mobile phone number must be provided in order for test results to be pushed electronically to the kiosk user. In addition, although a touch screen 206 is described, kiosk 1 may include alternative or additional user input mechanisms including, without limitation, keyboard, mouse or voice command input mechanisms.

If payment is required, the kiosk uses card reader 201 when prompted by touch screen 206 to scan payment information. Touch screen 206 and card reader 201 are connected to computer 203 that controls the transaction with online third-party verification software accessed through Internet connection hub 204 or through a direct Internet connection.

As shown in FIG. 2, blood/fluid analyzer 101 may be connected to hub 204 or to computer 203 using a wired connection (serial, USB or proprietary connector) or a wireless connection (WiFi, Bluetooth, ANT+ or any other wireless protocol). Hub 204 and/or computer 203 securely transmit the test measurement results from blood/fluid based test devices 100 along with personal health information obtained from the user, to cloud storage 2 or the cloud for storage and/or further user consultation. Cloud storage 2 may include databases of useful information such as payers, healthcare providers, advertisers, and health records.

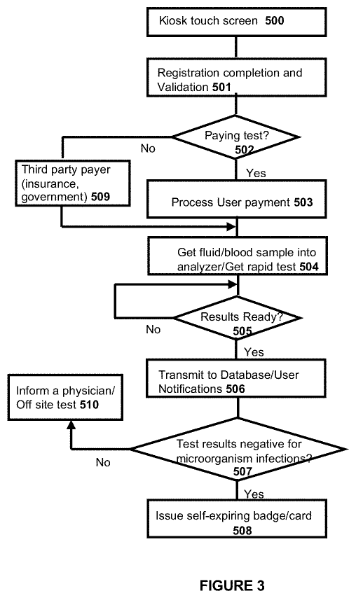

Kiosk software 205 guides the interaction between kiosk 1 and the kiosk user. Kiosk software 205 implements an algorithm that is depicted by the flowchart of FIG. 3. Kiosk software 205 takes the form of computer-readable program code embodied in a non-transitory computer-readable medium that is executed by a processor of kiosk computer 203 and/or cloud storage 2. In step 500, the kiosk user touches touch screen 206 in order to begin interacting with kiosk 1 or alternatively, the kiosk 1 will have motion sensors that can detect the user approaching the kiosk. In step 501, touch screen 206 displays a list of tests available to be performed by blood/fluid based test devices 100. The costs of each test may also be displayed. The kiosk user makes a selection of tests, for example, by check marking desired tests.

If a test selected by the kiosk user is a test that requires payment (step 502), then a process is initiated to verify the user and obtain payment (step 503). In particular, credit card information is obtained from the kiosk user for payment by inviting the user to swipe a credit card in card reader 201. If the insurance is paying for the test, then the user's insurance eligibility is checked and a co-pay amount, if any, is determined. If the government is paying for the test, then the user's eligibility is checked accordingly (step 509).

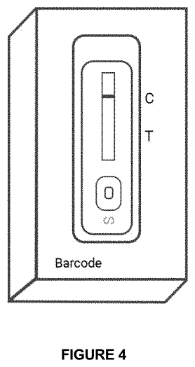

After payment is processed, the test procedure begins (step 504). If blood/fluid collection is required, the kiosk user uses collection device 103 to provide a blood/fluid sample to collector 102 by a finger prick, and collector 102 is provided to analyzer 101 to begin blood/fluid analysis. Attendant, if present, can assist the kiosk user in these operations. FIG. 4 shows an exemplary rapid test cassette that can be used in the present invention. The rapid test cassette will have an identifier such as a barcode to scan the device and link to the subject.

In step 506, the test results are aggregated and transferred to cloud storage 2 or the cloud, along with an electronic notification to the user. All the data, reports, pictures and videos are stored on the secure cloud. After this, the sample is moved to safe disposal and kiosk 1 is ready for the next kiosk user.

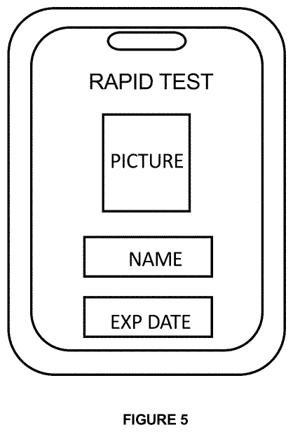

After the subjects are tested negative for selected microorganism infections (step 508), the kiosk distributes self-expiring badges to the subjects free of microorganism infections. For this, the kiosk 1 comprises a badge dispensing slot 301 through which the self-expiring badges are dispensed to subjects, a return area 302 through which the self-expiring badges are returned by subjects to the kiosk 1. Also internal to the kiosk is a badge storage area 304 and a transport mechanism. The badge storage area stores badges waiting to be dispensed to a subject and receives badges that are returned by subjects. The transport mechanism is designed to transport a badge from the storage area to the slot during the issuance of a badge and transport a badge from the slot to the storage area when the badge is returned. As a result, badges that have been returned directly to the kiosk for reuse and reissue of the badges. FIG. 5 displays an exemplary self-expiring badge of the invention.

The test results along with badge details and any other relevant information may also be provided by Cloud storage 2 and/or kiosk 1 to user device 3 via Internet connection 12. A mobile application executing on user device 3 may display the test data and test result history, and may provide other options such as sharing information with a medical network or a physician; storing data in a personal health record; and making an appointment with a physician, who will be able to access the recorded health record of the consumer. The application may also display advertisements of products and services that are of relevance to the user's health situation, as well as trends and progress made by the user.

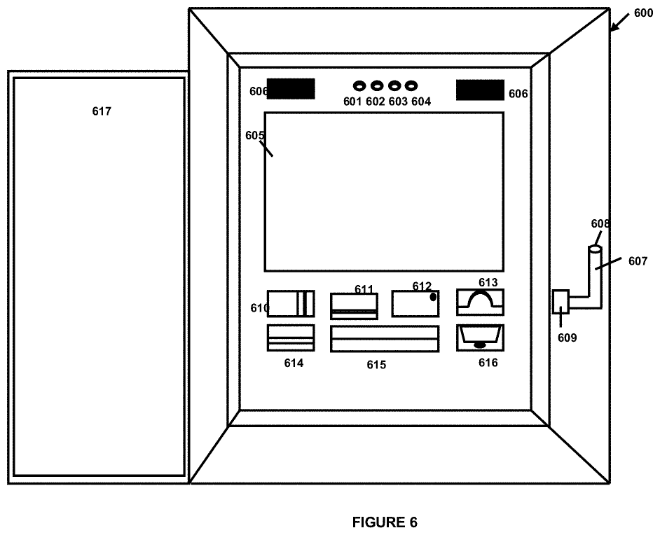

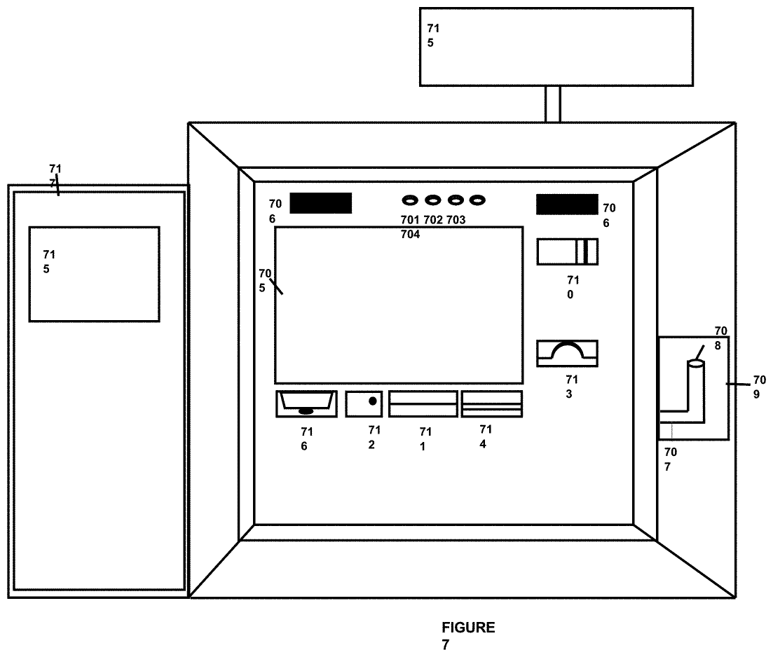

FIG. 6 and FIG. 7 shows the representative kiosks (600, 700) of the present invention. As the subject approaches the kiosk facility, the motion sensor (601, 701) detects the subject and boot up the system. The camera (602, 702) identifies and validates if the subject has already a registered user. A temperature sensor (603, 703) is used to measure the temperature of the kiosk user. A radar/sonar (604, 704) is used to detect the objects and the spacing. If the subjects are paying for the test, the credit card reader (610, 710) or a cash dispositor (614, 714) is used to collect the payment. If the subject orders a blood/fluid test, the subject may be provided with an alcohol swab through the disperser (615, 711) and the kiosks (600, 700) may include additional camera and a laser (not shown) to guide the user to place the finger at the right position in the finger slot (613, 713). A third camera records the rapid test process and display the process and the results on the touch screen (605, 705). FIG. 4 shows an exemplary rapid test cassette that can be used in the present invention. The kiosks can perform additional vital measurements as required by the tests selected by the user. For example, subject's oxygen saturation levels may be measured before the finger prick. Additionally, the dispenser (615, 711) is capable of providing respiratory masks to the users. Further, the dispenser (615, 711) can be used to provide additional items. For example, when required, the dispenser (615, 711) can provide a medical test kit comprising (a) a saliva collection kit for collecting a saliva sample; and (b) a mouth swab. In this scenario, the subject opens the kit and the mouth swab is used to swab the mouth or throat to collect a saliva sample or throat culture. The swab is placed into an envelop provided in the medical test kit and deposit (re-insert) in the dispenser. The saliva analysis may be performed at the kiosk or can be send to the off-side test lab.

The kiosks include a breathalyzer (607, 707) to determine the analytes in the subject's breath sample. The breathalyzer includes a hole (608, 708) to allow the subject to insert a tip. The tip is provided to the subject through the disperser (615, 711). The breathalyzer may be attached to a lever (609) such that the user can adjust the height of the breathalizer. Additionally, the breathalyzer can be placed in a house (709) and the subject can open the door to access the breathalyzer. All the data can be stored on the card in a magnetic strip, barcode, or any other suitable format for storing data. Kiosks are accompanied by display units (715) for advertisements of products and services that are of relevance to the user's health situation. After the test results are negative for microorganism infections, the subjects are provided with self-expiring badge through the ID badge output (611) or can be the dispenser (711). A typical self-piring badge is shown in FIG. 5.

Other aspects, objects and advantages of the present invention can be obtained from a study of the drawings, the disclosure and the claims.

1. A self-service kiosk for producing self-expiring badge comprising:
a customer interface with a computer system;
a collector for collecting a biological sample from a subject;
an analyzer for analyzing the biological sample; and
a self-expiring badge printer and output;
wherein said analyzer analyzes the biological sample for microorganism infections and wherein said self-expiring badge is issued to said subject upon said subject tests negative for microorganism infections.

2. The self-service kiosk of claim 1, wherein the customer interface with a computer system provides test results of analyzing the biological sample.

3. The self-service kiosk of claim 1, wherein the microorganism is a virus and said self-expiring badge is issued to said subject upon said subject tests negative for viral infections.

4. The self-service kiosk of claim 1, wherein the microorganism is a bacterium and said self-expiring badge is issued to said subject upon said subject tests negative for bacterial infections.

5. The self-service kiosk of claim 1, wherein the microorganism is a yeast and said self-expiring badge is issued to said subject upon said subject tests negative for yeast infections.

6. The self-service kiosk of claim 1, wherein the microorganism is a fungus and said self-expiring badge is issued to said subject upon said subject tests negative for fungal infections.

7. The self-service kiosk of claim 1, wherein said collector comprises a finger stick device for obtaining the biological sample from the subject.

8. The self-service kiosk of claim 1, wherein the biological sample is blood.

9. The self-service kiosk of claim 1, wherein the expiration of the self-expiring badge is visually observed.

10. The self-service kiosk of claim 9, wherein the self-expiring badge includes an area wherein the expiration date of the badge is written.

11. The self-service kiosk of claim 1, wherein the self-expiring badge is tampering resistant.

12. The self-service kiosk of claim 1, wherein the self-expiring badge is re-usable.

13. The self-service kiosk of claim 1, further comprising a breathalyzer for receiving and testing a breath sample from the subject.

14. The self-service kiosk of claim 1, further comprising a card reader for obtaining payment information from the subject.

15. The self-service kiosk of claim 1, further comprising an Internet connection hub for transmission of the test results to a cloud storage or a user device.

16. A non-transitory computer-readable medium storing program code that causes a processor to execute operations, at a self-service kiosk, comprising:
displaying tests available to be performed at the kiosk;
obtaining from a selection of a test from a subject;
obtaining payment from the subject and performing financial transactions;
performing the selected test;
providing results of the test to the subject; and
printing a self-expiring badge, wherein said self-expiring badge is printed upon said subject tests negative for microorganism infections.

17. The non-transitory computer-readable medium of claim 16, wherein the available tests include a biological sample analysis test that is performed automatically by collecting the biological sample from said subject.

18. The non-transitory computer-readable medium of claim 16, wherein the available tests further comprise a non-invasive test for vitals measurements.

19. The non-transitory computer-readable medium of claim 18, wherein the non-invasive test measures at least one of weight (body mass index), blood pressure, temperature, heart rate and oxygen saturation.

20. The non-transitory computer-readable medium of claim 16, wherein the program code causes the processor to execute a further operation comprising:
transmitting the results of the test to a mobile application on a user device.

21. The non-transitory computer-readable medium of claim 16, wherein the program code causes the processor to execute further operations comprising at least one of:
providing a guidance plan to the subject based on the test results; and
sharing the test results with subject's physician or any assigned physician.

22. A method for identifying subjects free of microorganism infections using a kiosk comprising the steps of:
displaying instructions for using the kiosk;
collecting a blood sample from the subject;
testing the blood sample for the microorganism infections and producing a result; and
storing the result for subsequent retrieval and transmission; and
issuing a self-expiring identification badge upon said subject tests negative for microorganism infections.

23. The method for determining microorganism infections in the subject as set forth in claim 22, wherein using the kiosk includes the step of registering and authorizing the subject to access the kiosk.

24. The method for determining microorganism infections in the subject as set forth in claim 22, wherein expiration of the self-expiring badge is visually observed.

25. The method for determining microorganism infections in the subject as set forth in claim 22, wherein the self-expiring badge includes an area wherein the expiration date of the badge is written.

26. The method for determining microorganism infections in the subject as set forth in claim 22, wherein the self-expiring badge is tampering resistant.

27. The method for determining microorganism infections in the subject in claim 22, further comprising the step of capturing a digital still photograph of the subject interacting with the kiosk.
